(19) United States
(12) Patent Application Publication
(10) Pub. No.: US 2005/0260592 A1
(43) Pub. Date: Nov. 24, 2005

(54) Method for performing high-throughput analyses and device for carrying out this method

(76) Inventors: Walter Gumbrecht, Herzogenaurach (DE); Manfred Stanzel, Erlangen (DE)

Correspondence Address: HARNESS, DICKEY & PIERCE, P.L.C., P.O.BOX 8910, RESTON, VA, 20195, US

(21) Appl. No.: 10/522,001

(22) PCT Filed: Jul. 21, 2003
(86) PCT No.: PCT/DE03/02444

§ 371 Date: Jan. 21, 2005

(30) Foreign Application Priority Data
Jul. 22, 2002 (DE) 102 33 212.6

(51) Int. Cl. C12Q 1/68; G06F 19/00; G01N 33/48; G01N 33/50

(52) U.S. Cl. 435/6; 702/20

(57) ABSTRACT

A method is for performing a high-throughput analysis, according to which processing simultaneously ensues in a continuous manner at a number of work stations. In order to improve the sample throughput, a support, which includes a multitude of spots, particularly a number of spot arrays, is used that is moved in a time manner through the work stations. The corresponding device includes a bio-chip arrangement having a multitude of spots, particularly a number of spot arrays that are arranged in an interspaced manner on a common support made of a flat material.

This application is the national phase under 35 U.S.C. § 371 of PCT International Application No. PCT/DE03/02444 which has an International filing date of Jul. 21, 2003, which designated the United States of America and which claims priority on German Patent Application number DE 102 33 212.6 filed Jul. 22, 2002, the entire contents of which are hereby incorporated herein by reference.

### FIELD OF THE INVENTION

The invention generally relates to a method for performing high-throughput analysis and/or an associated device for carrying out the method, respectively. High-throughput analysis is known as the term “HTS” (=High Throughput Screening) in biochemical analysis.

### BACKGROUND OF THE INVENTION

A conventional—optically readable—biochip includes a miniaturized carrier, to the surface of which an array of extremely small quantities of substance, so-called spots, is applied. The spots contain probe molecules immobilized on the carrier surface, usually nucleotides having up to approximately 30 bases (DNA chip).

In the course of an analytical examination, a sample liquid containing nucleic acids with an optically active label, so-called target molecules, is applied to the spot array. Target molecules that are complementary to the probe molecules with regard to their base sequence attach thereto (hybridization). After the removal of non-hybridized target molecules, the result of the hybridization can be read out optically on the basis of the label of the target molecules.

Analysis methods of this type are used for example in the development of medicaments, in pharmacology and pharmacokinetics for researching the effect and side effect of medicaments, in diagnosis for identifying pathogens and for determining medicament resistances, and also in foodstuffs inspection for identifying food-stuffs altered by genetic engineering. Conventional analysis methods use biochips disclosed in WO 00/73504 A2, by way of example, in which a single spot array is present on a slide-sized carrier.

In order to carry out HTS analyses, it is often necessary, on account of the high number of individual determinations or hybridizations, for very many biochips to be prepared, subjected to data acquisition and stored in a supply container. Furthermore, each individual biochip has to be transported to an analysis and detection device, where sample liquid is added to it. After a reaction time has elapsed, a rinsing step is effected, by which the sample liquid is removed again. The analysis result is then detected and read out and the used biochip is finally removed from the analysis and detection device. A multiplicity of time-consuming manipulations are thus required.

In addition, WO 00/63705 A1 discloses an arrangement and a method for the transfer of small substance volumes, in which, in a continuous run, the individual spots of a biochip are furnished with reagents in a precise manner in respect of location by means of a suitable arrangement. In detail, for this purpose pipettes that can be moved three-dimensionally are present at a distance above a continuously running tape. The pipettes draw different volumes of liquid from different supply containers via perforations in a running tape and deposit them at the individual spot points of a chip. No statements are made here about carrying out measurements with biochips furnished in such a way.

### SUMMARY OF THE INVENTION

It is an object of an embodiment of the invention to provide a method for performing high-throughput analyses and/or to provide a device suitable therefor. In this case, it is an aim, in particular, to reduce the number of manipulation steps required and thus the time spent for high-throughput analyses.

An object may be achieved by a method and/or a device having a biochip arrangement. Developments and other exemplary embodiments of the method and of the associated device are specified in the disclosure hereafter.

In the method according to an embodiment of the invention, individual work steps are carried out simultaneously on the cyclically moved carrier. A work step for supplying the measurement sample to the measurement spot and a work step for measurement with supply and removal of liquid are necessary at the very least. Further work steps include temperature regulation and/or air conditioning and, if appropriate, reaction residence times. It is thus possible to establish in a targeted manner the measurement parameters “temperature” and/or “moisture”, on the one hand, but also the influencing variables “type and flow” of the reagents used, on the other hand.

According to an embodiment of the invention, a device having a biochip arrangement having a plurality of spot arrays arranged on a common carrier is used for carrying out a high-throughput analysis. Conventional HTS analyses, by contrast, use carriers on which only a single spot array is present. In order to carry out a test, the carrier—usually via a robot arm—is taken from a magazine and supplied to an analysis and detection device. After the test has ended, the carrier is removed therefrom and disposed of. By virtue of an embodiment of the invention, by contrast, a multiplicity of tests are possible with only a single sequence of the manipulation steps mentioned. The time spent for a test series can therefore be considerably reduced.

In particular on account of a flat embodiment according to the invention, it is also possible to save material and volume by virtue of only the spot arrays being situated on the flat carrier, but no devices whatsoever for volume separation such as e.g. plastic cavities, through-flow channels or closure covers. The aforementioned devices are then placed onto the flat carrier in a reusable manner at the system end.

The multiplicity of spot arrays present on a carrier requires that an individual spot array or a group of spot arrays of identical type can be subjected to a test independently of other spot arrays. This is made possible by virtue of the fact that at least one spot array is enclosed by a hollow body that produces a spatial separation from other spot arrays. Manipulations can then be performed within the space thus created, for example a specific sample solution can be added to a spot array or a group of spot arrays without the rest of the spot arrays present on a carrier being impaired thereby.

A spatial separation of the aforementioned type can be accomplished in a manner that is technically simple to realize by virtue of a hollow body being placed onto the carrier in such a way that it surrounds at least one spot array in sealing fashion with a peripheral wall. In this way, it is possible, by way of example, to create a space that serves for air conditioning of the gas phase present above a spot array. It is also possible to effect a plurality of spatial separations simultaneously in order to treat individual spot arrays or groups of spot arrays differently. Moreover, a further time saving can be achieved by way of such parallel treatment.

Generally, the sample liquid brought into contact with a spot array is removed again after the reaction or hybridization has ended. This method step can also be realized in a manner that is simple in terms of method technology by way of a spatial separation of the type outlined. The hollow body merely has to be configured in such a way that a rinsing liquid can be conducted through its internal space. By using a hollow body configured in this way, reagent solutions can also be conducted over the spot array.

With regard to the space requirement in a magazine and its manipulability, a carrier is advantageous which is essentially formed from a flat material, for instance a plastic film. Such carriers can be arranged in a magazine with a small space requirement and be isolated from the surroundings for the purpose of relatively long storage. The use of a tape-type carrier made of a flexible material is especially advantageous.

Such a carrier can be stored in the form of a roll in a magazine, be continuously removed from said magazine, passed through an analysis and detection device and subsequently be wound up again to form a roll or be supplied for disposal in the form of sections. It is particularly advantageous to use a carrier format having a width of 35 mm with a two-row perforation as is used in the film industry or else already as a carrier of chips in semiconductor technology. A cyclic advancing movement is also conceivable in addition to continuously transporting the carrier tape through an analysis and detection device. During the standstill times, manipulations can then be performed without any problems on the carrier or on the spot arrays situated thereon.

In the context of the invention, biochips may be realized on the carrier in different ways, in principle. It is conceivable, for example, for the spot arrays to be applied directly to the carrier material, whereby a biochip is already defined. An optical read-out of the test results is appropriate in the case of this type of realization.

Particularly when using a carrier tape with electrical components, such as e.g. metal layers, an electrical detection of the test results is advantageous because it can be integrated into an analysis method that works continuously or cyclically more easily than an optical detection. The individual spots of the spot arrays can then be realized e.g. directly in small cavities of the carrier. For this purpose, the flat carrier may include e.g. laminated layers of at least one insulation layer and at least one metallic layer. An insulation layer has openings in partial regions, thus giving rise to cavities, which is open on one side and closed on the opposite side by at least one metal layer and, if appropriate, a further insulation layer.

The microcavities with a diameter of a few 100 μm that are realized in this way then serve as a receptacle for the spot-specific probe molecules (e.g. DNA catcher oligonucleotides). Each spot is then contact-connected to at least one metal area that serves as an electrode.

In another realization of an embodiment of the invention, the spot arrays are applied to chips, e.g. silicon chips, and, for their part, mounted on a carrier material. In the case of electrical measurement, the electrical signals can be tapped off directly from the chip or advantageously be passed via an electrical intermediate connection (e.g. thin bonding wires) between chip and metal layer of the carrier, the electrical intermediate connection being fixed upon production, and be read out via a temporary electrical contact between carrier metalization and read-out unit.

For method control purposes, it is expedient if data providing information about the type and number of the spot arrays situated on the carrier and about the method steps necessary for a specific analysis aim are present on the carrier. The data are preferably stored on at least one additional memory chip (e.g. EPROM).

Many analysis tasks require the spot arrays to be cooled or heated. In the replication of DNA by PCR (Polymerase Chain Reaction), by way of example, cooling and heating are done for the purpose of thermocyclization. Particularly in the case of carriers based on flat material, this can be realized in a simple manner if heat is supplied or heat is dissipated from the rear side region of the carrier opposite to a spot array. On account of the use of small material thicknesses (e.g. 50 μm), material areas (a few mm²) and materials having high thermal conductivity (e.g. copper, gold), extremely fast and at the same time energy-saving temperature changes or regulations can be realized in conjunction with an extremely small heat capacity. This is preferably realized by way of an areal contact with a coolable or heatable body.

Carrying out the analyses requires reagents, which can be pumped over the respective spot array via suitable hollow bodies e.g. in the form of through-flow arrangements.

A biochip arrangement for carrying out the method described also has the following advantageous features in addition to those already described in connection with the analysis method:

The spot arrays are arranged in a depression of the carrier or within an elevation e.g. in the form of a polymer ring having a height of a few 100 μm, thereby facilitating application of sample liquid to a spot array. The depression prevents sample liquid from being able to reach adjacent spot arrays if appropriate with utilization of surface tension effects.

In principle, spot arrays may be present on both sides of a carrier. However, since sample liquid has to be applied to the spot arrays, it is expedient for the latter to be arranged only on one side, namely that side of the carrier which faces upward when carrying out the analysis. The rear side is then available for a transfer of heat through areal contact. In the case of electrically readable biochips, there is sufficient space available on the rear side or underside of the carrier for the arrangement of electrical contact areas and contact elements interacting with the latter.

All the devices for application of liquid, electrical contact-making, thermostatic control, air conditioning and also for fluidic contact-making of rinsing and reagent solutions can be moved perpendicular to the tape running direction in order to enable the tape to be freely transported further.

### BRIEF DESCRIPTION OF THE DRAWINGS

Further details and advantages of the invention emerge from the following description of figures of an exemplary embodiment with reference to the drawings in conjunction with the patent claims. In the figures:

FIG. 1 shows a plan view of a biochip arrangement,

FIG. 2 shows the detail II from FIG. 1 in an enlarged illustration,

FIG. 3 shows a cross section corresponding to line III-III in FIG. 2,

FIG. 4 shows a plan view of a differently configured biochip arrangement,

FIG. 5 shows a schematic illustration of a device for carrying out an HTS analysis method,

FIG. 6 shows an enlarged detail from FIG. 5,

FIG. 7 shows an alternatively configured device in an illustration corresponding to FIG. 5, and

FIG. 8/9 show the cross sections of carrier tapes with directly applied spots.

### DETAILED DESCRIPTION OF THE EXEMPLARY EMBODIMENTS

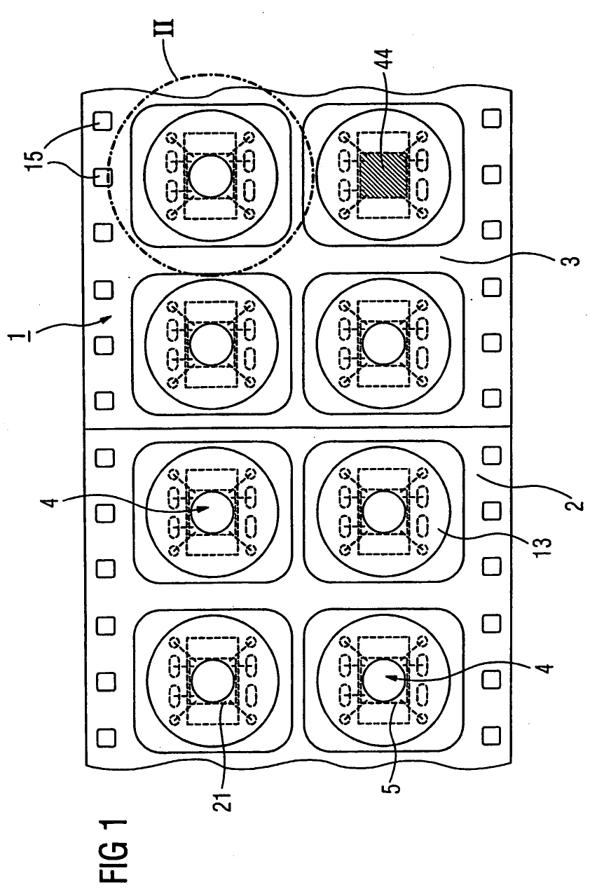

FIG. 1 shows a biochip arrangement 1. The latter includes a carrier 2 made of a flat material, for example made of a plastic film, and biochips 4 arranged on one side thereof, the analysis side 3. In the present example, a total of 8 biochips are arranged in two parallel rows extending in the longitudinal direction of the carrier 2. In principle, however, an arbitrary arrangement and number of the biochips 4 are possible. In particular, the carrier 2 may be made significantly longer, namely in the form of a flexible tape, as will be explained further below.

In the case of the exemplary embodiment illustrated in FIG. 1 to 3, the biochips 4 are electrically readable. They include a silicon chip 5, which is produced in a conventional manner and bears by one side, its flat side, on the analysis side 3 of the carrier 2. An electrically conductive layer 7 made, for example, of copper is applied to the rear side 6 of the carrier 2 opposite to the analysis side 3. Grooves 8 subdivide the layer 7 into contact areas 9. Each silicon chip 5 is assigned a group of contact areas 9. The contact areas 9 are electrically connected to the silicon chip with the aid of wires 10, so-called bonding wires. In order to make this possible, cutouts 31 by which the electrically conductive layer 7 is accessible are present in the carrier 2. Further variations are possible in addition to this configuration of the biochip arrangement 1. By way of example, fixing the silicon chip according to the so-called flip-chip technology is conceivable.

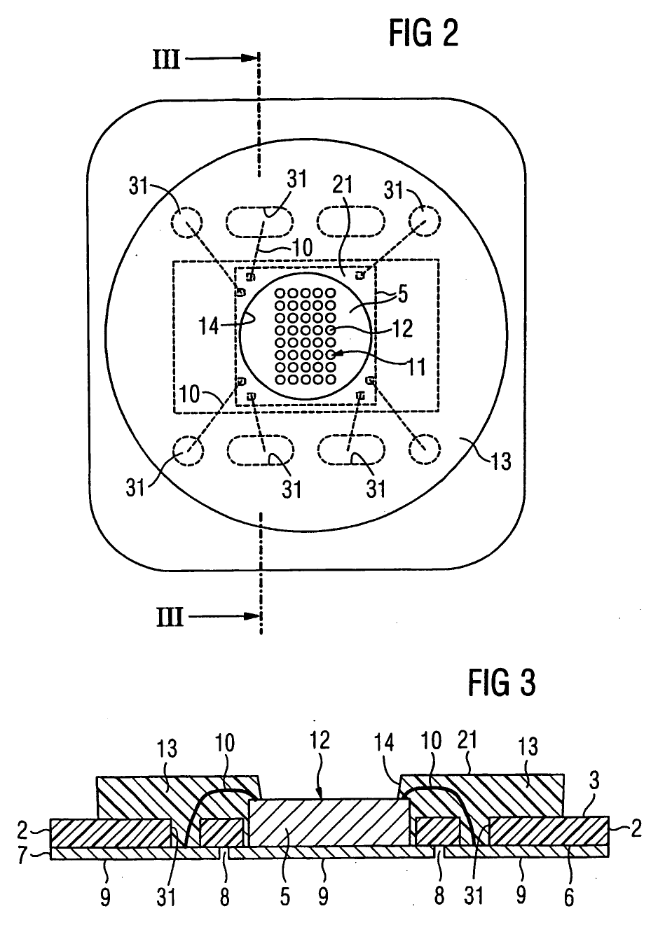

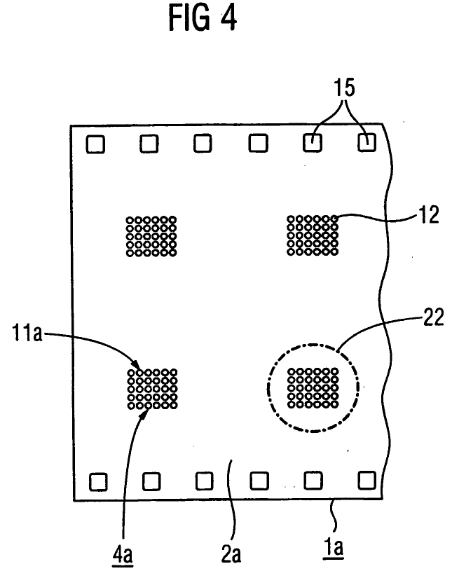

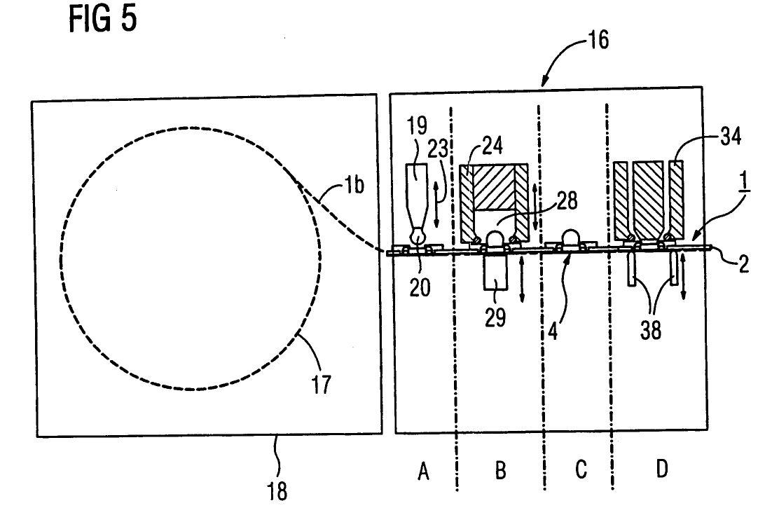

A spot array 11 of microdroplets or spots 12 is applied on the side of the silicon chip 5 facing away from the layer 7. The spots contain probe molecules, in particular nucleotides having a few up to 40 bases. Only a few spots 12 are illustrated in FIGS. 2 and 4 for graphic reasons. In reality, significantly more spots 12 can be accommodated on a silicon chip. The area regions of the silicon chip 5 that are arranged below the spots 12 are electrically sensitive regions with interdigitated electrodes, which is not illustrated in FIG. 2.

In a simplified illustration, the electrically readable biochips 4 outlined work e.g. as follows: probe molecules present in the spots 12 are hybridized with target molecules carrying a label, e.g. biotin. By way of a rinsing process with a reagent solution containing so-called enzyme conjugate (e.g. streptavidine-labeled alkaline phosphatase), target molecules not coupled to the probe molecules are removed and, at the same time, the enzyme “alk. phosphatase” is bound to the probe/target molecule hybrid. Finally, by rinsing with a suitable enzyme substrate, e.g. p-aminophenyl phosphate solution, p-aminophenol is formed here in a manner catalyzed enzymatically, and can be detected electrochemically at the electrodes.

The silicon chip 5 is embedded in an encapsulating composition 13 for the purpose of fixing to the carrier 2 and for the purpose of mechanical protection. A cutout 14, that frees the spot array 11 is present in the top side 21 of the encapsulating composition 13. The carrier 2 has a perforation 15 on both sides, which extends in longitudinal direction 15, and a width of 36 mm. It thus has the format of a 36 mm roll film known from photography.

Such a format is used in the production of chip modules for smart cards. Therefore, a biochip arrangement 1 is produced by resorting to this technology or the devices provided therefor for processing the carrier 2 (e.g. lamination of insulating and electrically conductive layers) etc.

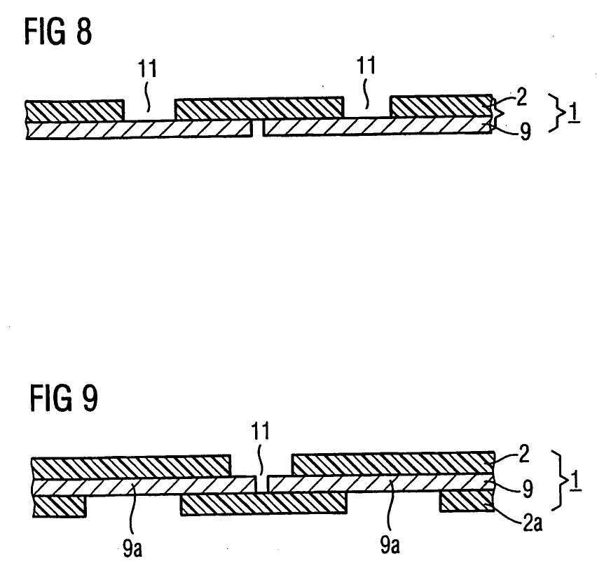

Instead of electrically readable biochips 4, a carrier 2a may also be populated directly with spot arrays 11a in accordance with FIG. 4 which are optically or electrically readable (FIG. 4), which will be discussed further below. In detail, for this purpose, spot arrays 11a are introduced onto the carrier 2a directly or e.g. in microcavities (FIGS. 8 and 9). A biochip 4a is then composed of a spot array 11a and a region 22 of the carrier 2a assigned thereto.

For the application of the spot arrays 11a, it is possible in this case, as also in the case of electrically readable biochips 4, to use known ink jet printing methods. In the case of the biochip arrangement 1a, too, a perforation 15 on both sides may be expedient during the production process and—as also in the case of the biochip arrangement 1 described above—for transport during an HTS analysis.

It is illustrated with reference to FIGS. 5 to 7 that, by way of a fixed assignment of individual biochips with measurement spots to the common carrier, the HTS analysis can be carried out in separate work steps A to D simultaneously at different spots. As a result of the carrier 2 being advanced cyclically, the individual spots or biochips successively run through the individual stations A to D. The working speed can be influenced by prescribing a suitable advancing cycle.

An analysis and detection unit, called analysis unit 16 for short hereinafter, illustrated in a highly simplified manner in FIG. 5 is used for carrying out an HTS analysis. A biochip arrangement 1, 1a, 1b is introduced into the analysis unit 16 and the spot arrays situated thereon are processed in accordance with the analysis. In the case of the exemplary embodiment shown in FIG. 5, a biochip arrangement 1b configured in the form of a flexible tape is employed.

The tape is constructed like the biochip arrangement 1 shown in FIG. 1. It includes spot arrays 11 having properties required for the respective examination and is wired up to form a roll 17 which is accommodated in a protective magazine 18. The tape-type biochip arrangement 1b is transported through the analysis unit 16 for which purpose the perforation 15 on both sides is useful. Within the analysis unit 16, firstly a sample liquid 20 is applied to one or a plurality of biochips 4 with the aid of a dispensing device 19. The depression or cutout 14 present in the encapsulating composition 13 prevents the sample liquid 20 from being able to flow away laterally and reach other biochips 4 or spot arrays 11.

The dispensing device 19 is expediently embodied in the form of a pipette. If necessary, a plurality of such pipettes may be used in parallel in order, for instance, to add sample liquid 20 to a group of spot arrays 11. The dispensing device 19 is guided movably in the analysis unit 16 orthogonally to the chip arrangement 1 in accordance with the double arrow 23 and can be charged with different sample liquids.

In many hybridization reactions or other reactions that can be used for the analyses mentioned in the introduction, a relatively long reaction duration is required. During the reaction duration there is the risk of the very small quantity of sample liquid at least partly evaporating and, as a result, the concentration ratios changing in the sample liquid 20. The situation in which CO₂ or other gases from the air dissolve in the sample liquid 20 also cannot be precluded.

For this reason, the gas phase above a biochip 4 is air-conditioned. For this purpose, an approximately cylindrical hollow body 24 is placed onto the biochip arrangement 1b in such a way that it surrounds at least one spot array 11 in sealing fashion with a peripheral wall 25.

For this purpose, a sealing ring 26 is fitted to the end side of the hollow body 25 facing the chip arrangement 1 and bears in sealing fashion on the top side 21 of the encapsulating composition 13, said top side being formed as a planar area. The hollow body 24 is sealed with respect to the atmosphere at the top side by way of a molding 27. A chamber 28 is enclosed between the hollow body 24 and the biochip 4 interacting therewith. Said chamber 28 has a volume that permits evaporation of sample liquid 20 at most only to an inconsiderable extent. Moreover, a microclimate that prevents evaporation can be maintained in the chamber 28.

Many reactions require cooling or heating. This may be accomplished with the aid of a heated or cooled body 29 made of thermally conductive material which is brought into areal contact with the underside 30 of the chip arrangement 1b or the electrical contact areas 9 present there. The body 29 and also the hollow body 24 may be guided movably orthogonally to the biochip arrangement 1b (double arrows 32 and 33).

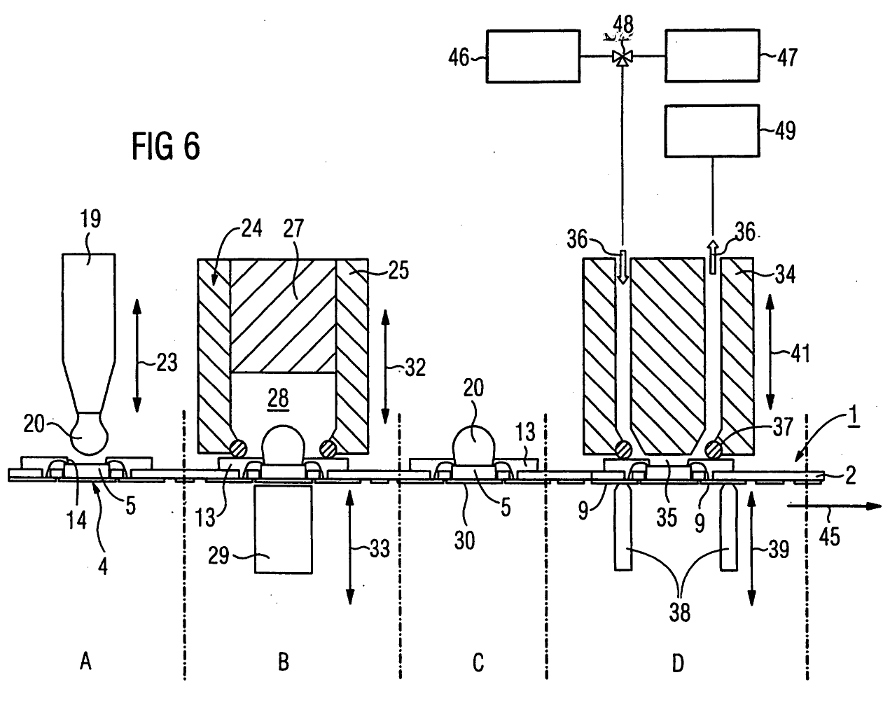

After the reaction residence duration has elapsed, the sample liquid 20 is removed. A second hollow body 34 may be used for this purpose, a rinsing liquid or reagent liquid being conducted through the internal space 35 of the second hollow body, as is indicated by the flow arrows 36 (FIG. 6). For this purpose, containers 46 and 47 may be present, which are connected via a valve 48 to the supply line for the internal space.

What is essential is that it is possible to bring, if appropriate successively, different reagents alternately with rinsing liquid to the measurement spots, a container 49 for receiving used liquid being present. Equally, an arrangement (not shown here) for temperature regulation of the measurement location may once again also be present. Defined changes in potential can thus be detected at the electrodes.

In order to prevent rinsing/reagent liquid from reaching adjacent biochips 4, the second hollow body 34 is also equipped with a sealing ring 37 on the end side, the sealing ring bearing on the top side 21 of the encapsulating composition 13 in sealing fashion. The hollow body 34 is likewise guided movably in a direction running orthogonally to the biochip arrangement 1 (double arrow 41). After or else during rinsing with the aid of the hollow body 34, the analysis result is electrically detected with the aid of at least two electrical taps 38, which make contact with at least two of the contact areas 9 assigned to a biochip 4 and which are guided movably orthogonally to the biochip arrangement 1 (double arrow 39). The hollow bodies 24 and 34 and also further hollow bodies (not illustrated) may also be used for purposes other than those mentioned above.

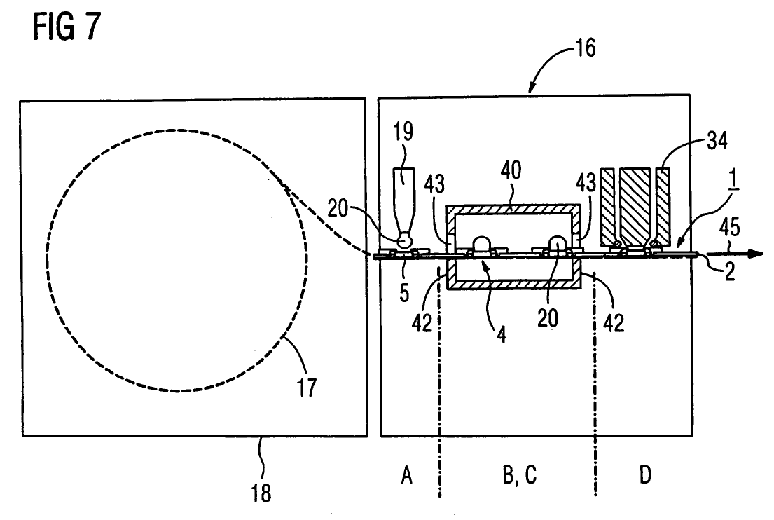

FIG. 7 illustrates an exemplary embodiment in which air conditioning of the gas space situated above one or a plurality of biochips 4 is realized by way of a hollow body 40 extensively enclosing the chip arrangement 1. Only at the front and rear end sides 42 facing in and counter to the advancing direction 45 of the biochip arrangement 1 is an opening 43 respectively provided in order to be able to transport the chip arrangement 1 through the hollow body 40.

The implementation of the method is generally facilitated by virtue of the fact that data concerning the type and positioning of the spot arrays 11, 11a and also further analysis-specific data are present on a biochip arrangement 1, 1a or on a carrier 2, 2a. In the case of a biochip arrangement corresponding to FIG. 4, this may be realized by use of a barcode (not illustrated). In the case of a biochip arrangement 1 with electrically readable biochips 4, a silicon memory chip 44 (FIG. 1) is expediently used.

FIGS. 8 and 9 specify alternatives to FIGS. 1 to 3 in which the carrier tape directly has individual measurement spots and thus as it were self-forms the biochip 1. In detail, insulator layers 2 and conductor layers 9 with individual perforations, which in each case form a spot 11, are present in FIG. 8. An arrangement is formed including two layers with a respective electrode per spot, which enables a measurement at the spot 11.

In FIG. 9, a biochip arrangement 1 is formed from three layers with in each case two electrodes per spot. Two insulator layers 2 and 2a and a conductor layer 9 are present in this case. It is thus possible, in principle, to carry out the same measurements at the measurement spot 11 as in FIGS. 5 to 7.

All the embodiments of the device make it possible to realize significantly improved HTS analyses with regard to the efficiency and, in particular, sample throughput, as has been described in detail above.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for performing a high-throughput analysis, in which samples are analyzed in a continuous manner and in which biochips with a multiplicity of measurement spots are used, comprising:
applying a measurement liquid to the spots or biochip situated on a carrier;
analyzing the samples of measurement liquid, wherein
the applying and analyzing are effected simultaneously at different spots or biochips, and wherein the carrier is moved to permit a continuous measurement at a speed determined by a movement cycle of the carrier.

2. The method as claimed in claim 1, wherein at least one of temperature regulation and air conditioning of the measurement liquid samples is interposed between the applying and analyzing.

3. The method as claimed in claim 2, wherein the air conditioning, if performed, serves as residence time of the measurement sample on the biochip.

4. The method as claimed in claim 1, wherein temperature regulation is effected following the sample application.

5. The method as claimed in claim 1, wherein at least one spot array is enclosed by a hollow body in order to create a spatial separation from other spot arrays.

6. The method as claimed in claim 5, wherein the hollow body is placed onto the biochip arrangement in such a way that it surrounds at least one spot array in sealing fashion with a peripheral wall.

7. The method as claimed in claim 5, wherein the hollow body serves for air conditioning of the gas phase present above a spot array.

8. The method as claimed in claim 6, wherein a rinsing liquid is conducted through an internal space of the hollow body.

9. The method as claimed in claim 5, wherein the carrier is one made of a flat material.

10. The method as claimed in claim 9, wherein a biochip arrangement with a tape-type carrier made of flexible material is used.

11. The method as claimed in claim 10, wherein the tape-type carrier is unwound from a roll and transported through an analysis unit.

12. The method as claimed in claims 1, wherein the carrier is one populated with electrically readable biochips.

13. The method as claimed in claims 1, wherein the carrier is one on which analysis-specific data are present.

14. The method as claimed in claims 1, wherein, for temperature control of a spot array or a reaction that takes place there, heat is supplied or dissipated from the rear side region of the carrier opposite to the array.

15. The method as claimed in claim 14, wherein, for the purpose of supplying heat or dissipating heat, the rear side region is brought into areal contact with a coolable or heatable body.

16. A device for analyzing samples in a continuous manner and in which biochips with a multiplicity of measurement spots are used, comprising:
a carrier wherein the biochips are arrangeable at a mutual distance on the carrier, the carrier being movable in a determinable cycle;
means for supplying a measurement liquid to the spots or biochips on the carrier; and
means for analyzing the samples of measurement liquid, wherein the applying and analyzing are effected simultaneously at different spots or biochips.

17. The device as claimed in claim 16, wherein the spot arrays are arranged in a depression.

18. The device as claimed in claim 16, wherein data for analysis control and data concerning the type and position of the spot arrays are present on the carrier.

19. The device as claimed in claim 18, wherein the data are stored in at least one memory chip.

20. The device as claimed in claim 16, wherein the carrier is essentially formed from a flat material.

21. The device as claimed in claim 20, wherein the carrier is formed as a flexible tape.

22. The device as claimed in claim 16, wherein the biochips are electrically readable biochips, each including a spot array and electrical contact areas.

23. The device as claimed in claim 22, wherein the spot arrays and the contact areas are arranged on different sides of the carrier.

24. The device as claimed in claim 22, wherein the biochips are embedded in an electrically insulating encapsulating composition, a cutout that frees the spot array and forms a depression being present in the encapsulating composition.

25. The device as claimed in claim 24, wherein a top side of the encapsulating composition that encompasses the cutout is formed as a planar area.

26. The device as claimed in claim 18, wherein the carrier includes a perforation extending in its longitudinal direction.

27. The device as claimed in claim 26, wherein the carrier includes a perforation on both sides and a width of 36 mm.

28. The method as claimed in claim 6, wherein the hollow body serves for air conditioning of the gas phase present above a spot array.

29. The device as claimed in claim 17, wherein data for analysis control and data concerning the type and position of the spot arrays are present on the carrier.

30. The device as claimed in claim 29, wherein the data are stored in at least one memory chip.

31. The device as claimed in claim 23, wherein the biochips are embedded in an electrically insulating encapsulating composition, a cutout that frees the spot array and forms a depression being present in the encapsulating composition.
